(19) United States
(12) Patent Application Publication
(10) Pub. No.: US 2024/0156237 A1
(43) Pub. Date: May 16, 2024

(54) Device for Grooming Tools

(71) Applicant: Dante Williams, Detroit, MI (US)

(72) Inventor: Dante Williams, Detroit, MI (US)

(21) Appl. No.: 18/452,402

(22) Filed: Aug. 18, 2023

Related U.S. Application Data
(60) Provisional application No. 63/371,793, filed on Aug. 18, 2022.

(51) Int. Cl. A45D 44/04; A61L 2/10

(52) U.S. Cl. CPC A45D 44/04; A61L 2/10

(57) ABSTRACT

A portable storage apparatus that attaches to a chair allows for the storage, organization, charging, and sanitation of grooming equipment. The storage apparatus includes a body defining a central cavity and having an opening to access the central cavity. At least a portion of the body is magnetic. The storage apparatus further includes a handle coupled to the body, a plurality of wheels coupled to the body, a movable lid coupled to the body and configured to cover the opening to the central cavity, a divider within the central cavity, a rechargeable battery attached to or integrated into the body, and a securement feature for securing the storage apparatus to a chair. The storage apparatus may further include a charging port and a sanitizer for respectively charging and sanitizing grooming tools.

### CROSS-REFERENCE TO RELATED APPLICATIONS

This application claims priority to U.S. Provisional Application No. 63/371,793 filed Aug. 18, 2022, which is hereby incorporated by reference in its entirety.

### BACKGROUND

### Technical Field

The disclosure is directed to the field of grooming services. More specifically, the present disclosure relates to a portable device that is attachable to a chair, allowing for the storage, organization, charging, and sanitation of grooming equipment, such as barber and salon equipment.

### Description of the Related Art

The COVID-19 pandemic has shown the importance of mobility and sanitation in modern American industry. Barber, salon, and grooming services are no exception to this general trend. As the haircutting industry prepares for a post-pandemic world, barbers need to have modern solutions to provide mobile services that also meet the stringent sanitation requirements of an industry that requires close contact with the face and body. Unfortunately, current products on the market do not offer barbers the ability to ensure their equipment is conveniently organized, charged, and accessible while also staying properly sanitized and ready for use.

Current products provide only partial solutions. Products exist that allow barbers to mobilize equipment, but they are cumbersome solutions designed for storage and transport rather than practical use. There are large cases that serve as storage units for a barber's tools, but they take up lots of space and are inconvenient to access when a barber is actively providing services. Mobile carts that include sanitizing features are even more cumbersome and less practical for active use.

Portable products that sanitize and charge equipment have been contemplated by others, but many use liquid or air sanitizing technology, creating noise, or requiring extra effort. These products are also not designed for active use and storage during a session; they are generally products used in between clients. Small caddies that attach to barber chairs provide an alternative, but these products generally do not include charging stations and sanitizing features. Their primary purpose is to provide a holder for equipment while barbers are performing services.

### SUMMARY

A need exists for a tool organizing product in the grooming services industry that provides mobility, modern sanitizing methods, and practical use during and between sessions and in smaller spaces. The product needs to be small and provide secure storage during transport to adapt to providing mobile grooming solutions. A further need exists to allow this same product to function as a convenient organizer for tools while services are being performed. Finally, a need exists for a product that charges corded and/or cordless tools and ensures they are sanitized to modern standards. This disclosure relates to an improved device that fulfills these mobility, practicality, charging, and sanitation needs.

One aspect of the disclosure relates to a storage apparatus for grooming equipment. The storage apparatus includes a body formed of a plurality of walls, the body defining a central cavity and having an opening to access the central cavity. At least a portion of a first wall of the body is magnetic. The storage apparatus further includes a handle coupled to the body, a plurality of wheels coupled to the body, a movable lid coupled to the body and configured to cover the opening to the central cavity, a divider within the central cavity, a rechargeable battery attached to or integrated into the body, and a securement feature for securing the storage apparatus to a chair.

In some embodiments of the storage apparatus, the handle is accessible from a top side of the body. In some embodiments, the handle is retractable into a sheath on or in a rear wall of the body.

In some embodiments, the opening to the central cavity and the lid are on a top side of the body. In some embodiments, the wheels are positioned on a bottom side of the body.

In some embodiments, the divider is removable and positionable at different locations within the central cavity. The storage apparatus of some embodiments comprises a plurality of dividers, and the plurality of dividers and the plurality of walls together define a plurality of storage compartments for grooming tools within the central cavity.

In some embodiments, the battery is detachable. In some embodiments, the battery is positioned on a bottom side of the body.

The storage apparatus of some embodiments further comprises a sanitizer within the central cavity. The sanitizer of some such embodiments comprises a UV light.

In some embodiments, the storage apparatus also includes an electrical charging means disposed within said central cavity. The electrical charging means of some embodiments comprises one or more of: an electrical port and a wireless charging coil. In some embodiments, the storage apparatus includes an electrical port on a surface of the body or a surface of the rechargeable battery.

In some embodiments, the storage apparatus includes a storage pocket on an outer surface of the body. In some embodiments, the securement feature comprises at least one of: a bracket, a hook, and a strap.

In some embodiments, the first wall of the body comprises at least one of: a left side wall, a right side wall, and a front wall. In some embodiments, the magnetic portion of the first wall comprises a magnetic strip. In some embodiments, an entirety of the first wall is magnetic.

### BRIEF DESCRIPTION OF THE DRAWINGS

FIG. 1A depicts a rear right perspective view of one embodiment of a storage apparatus.

FIG. 1B depicts a front left perspective view of the storage apparatus of FIG. 1A.

FIG. 2 depicts a front left perspective view of the storage apparatus of FIGS. 1A and 1B.

FIG. 3A depicts a left perspective view of one embodiment of a storage apparatus.

FIG. 3B depicts a right perspective view of the storage apparatus of FIG. 3A.

FIG. 3C depicts a front perspective view of the storage apparatus of FIG. 3A.

FIG. 3D depicts a top perspective view of the storage apparatus embodiment of FIG. 3A.

FIG. 4A depicts a top right perspective view of one embodiment of a storage apparatus.

FIG. 4B depicts a top left perspective view of the storage apparatus of FIG. 4A, with a lid lifted to show a central cavity.

FIG. 5 depicts a front bottom perspective view of one embodiment of a storage apparatus.

FIG. 6A depicts a front view of one embodiment of a storage apparatus.

FIG. 6B depicts a right side view of the storage apparatus of FIG. 6A.

### DETAILED DESCRIPTION

Provided herein is a portable storage apparatus that will allow barbers, hairstylists, pet groomers, and others to take their tools anywhere they need to go. The storage apparatus includes internal compartments for safe and secure storage and transport of grooming tools. It further includes a rechargeable battery and a means of delivering power from the battery to grooming tools. When services are ready to be provided, the storage apparatus may attach to a chair, serving as a battery-operated workstation for equipment. The storage apparatus of some embodiments also features a sanitizing component, referred to herein as a sanitizer, for the sanitization of equipment. The sanitizer is powered by the rechargeable battery. The storage apparatus of some embodiments also includes a magnetic portion that allows for a quick switch and storage of blades, scissors, or other tools while the user is performing services. The storage apparatus of some embodiments features a retractable handle and wheels for ease of transport. When services are ready to be provided, the storage apparatus may attach to a barber chair or any other chair via a securement feature.

The storage apparatus includes a body formed of a plurality of walls each having an outer surface and an inner surface. The walls of the body define a central cavity. An opening is provided to facilitate access the central cavity. In various embodiments, the top of the storage apparatus features a movable lid that covers the opening. The movable lid may be fully removable or pivotable and configured to move between an open configuration and a closed configuration. The lid is movable into the open configuration to provide access to the internal cavity. In some embodiments, the internal cavity is divided into a plurality of storage spaces for several different sized tools, including for example, different sized clippers, combs, brushes, shears, and clips. The plurality of storage spaces is defined by the inner surface of the body walls and one or more dividers. The storage apparatus of some embodiments includes one or more movable dividers within the internal cavity that can be positioned at varying locations to change the size and number of storage spaces to increase versatility and personalization of the storage apparatus.

The rechargeable battery of some embodiments is removable from the body of the storage apparatus, for example, to recharge in a charging station. In other embodiments, the rechargeable battery is permanently affixed or integrated into the body of the storage apparatus, and the storage apparatus includes or is attachable to a power cord for recharging the battery. The rechargeable battery may be attachable to an inner surface of the storage apparatus body, for example, it may be attachable to the body of the storage apparatus within the internal cavity. In some embodiments, the battery is attachable to an outer surface, for example, an outer left side wall, a right side wall, or a bottom wall of the storage apparatus body. The battery may be attachable via clips, a snap fit, a slide fit, a magnet, or any other attachment feature known to those skilled in the art. In some embodiments, the battery is integrated into, and not reversibly removable from, the body of the storage apparatus.

The means of delivering power from the battery to the grooming tools includes one or more of: an electrical outlet or port, a USB port, a wireless charging coil or pad, or any other power delivery means known to those skilled in the art. In some embodiments, one or more charging ports are provided within the internal cavity, on an outer surface of the body of the storage apparatus, and/or on an outer surface of the rechargeable battery.

The sanitizer of some embodiments is an ultraviolet (UV) light. In other embodiments, the sanitizer may be an ozone generator or any other sanitizing component known to those skilled in the art. In some embodiments, the action of closing the lid initiates the sanitizer, thereby turning it on or initiating an active state. In other embodiments, a switch or button is provided to activate the sanitizer. In various embodiments, the sanitizer will only operate when the lid is closed.

The securement feature of various embodiments includes one or more of: a bracket, a hanger, a hook, and a strap. The securement feature may be adjustable in size to facilitate attachment to chairs of varying sizes and shapes. For example, a strap may be adjustable via a rachet system, a strap adjuster or adjustable buckle, a plurality of reversible fasteners at varying locations, or any other adjustment mechanism known to those skilled in the art. In some embodiments, the bracket is formed of at least two components that slide relative to each other so that the width of the bracket is adjustable; such a bracket may include a plurality of holes and a pin to hold the bracket together at the desired width. The bracket of some embodiments is a tension bracket. In some embodiments, the strap is elastic. In other embodiments, the strap is reversibly closable via one or more hook and loop fasteners, ties, snaps, buttons, buckles, or any other fastener known to those skilled in the art. Once attached to the chair, the storage apparatus serves as a workstation and battery-operated charging station for grooming tools.

The magnetic portion of some embodiments includes one or more magnetic strips on an outer surface of the storage apparatus. In other embodiments, the magnetic portion includes one or more walls (or portions thereof) of a body of the storage apparatus. The magnetic portion may be positioned on, integrated into, or comprise a left side wall, a right side wall, or a front wall of the body. The magnetic portion allows for an easily accessible storage option of magnetic and non-battery-operated equipment (e.g., clips, blades, and shears) during grooming services.

The storage apparatus of some embodiments also includes one or more other features that facilitate convenient transport and/or use of grooming tools. For example, in some embodiments, the storage apparatus includes one or more front pockets for storage of clippers, brushes, combs, clips, or other tools while a user is providing grooming services. In some embodiments, the storage apparatus additionally or alternatively includes one or more cord guides to keep cords organized during use of grooming tools. In some embodiments, an extendable or retractable hair tray is provided. The hair tray is parallel or relatively parallel to the floor and a top wall of the storage apparatus body. The hair tray may be pulled out from a slot within the body of the storage apparatus or it may be folded down. In use, the hair tray provides a smooth, easy-to-wipe surface on which cut hair clippings can fall, reducing the amount of hair that lands on the floor.

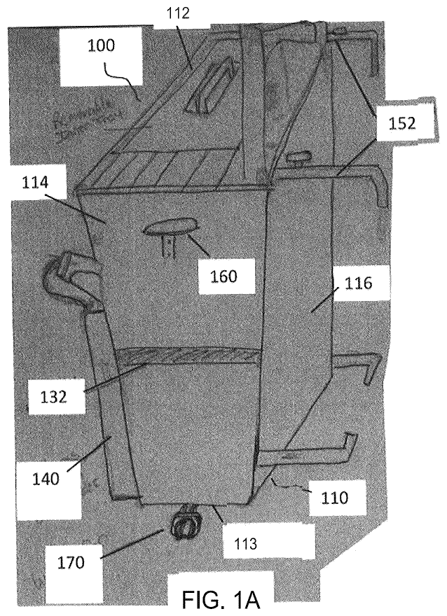

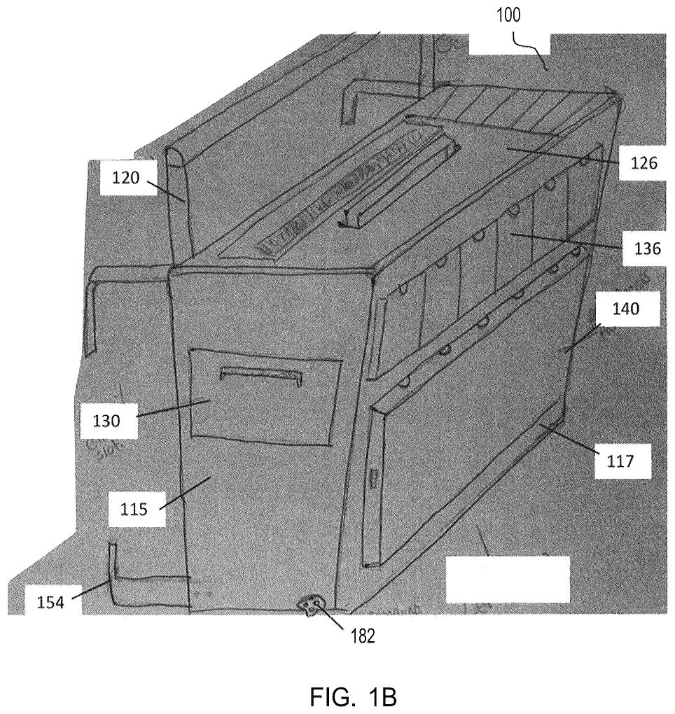

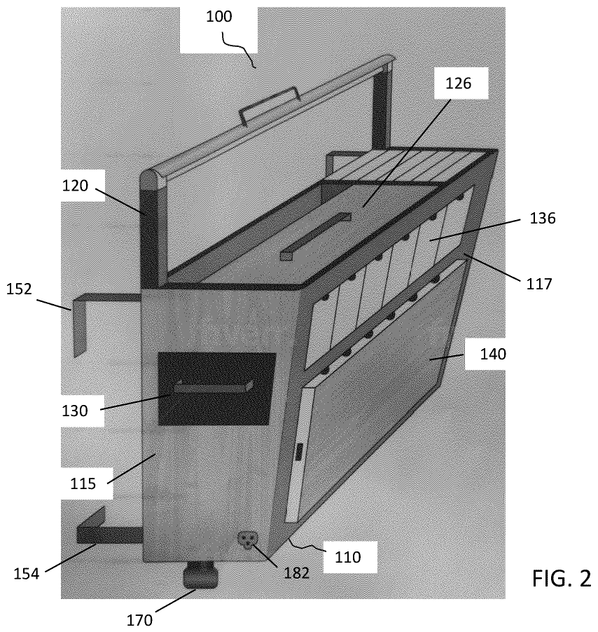

FIG. 1A, FIG. 1B, and FIG. 2 feature one embodiment of the storage apparatus 100. The storage apparatus 100 includes a body 110 formed of a top wall 112, right side wall 114, left side wall 115, back wall 116, front wall 117, and bottom wall 113. A top lid 120 is provided, which is movable about a hinge, to enable access to an opening that leads to an inner cavity 122. Within the opening is a removable inner tray 126 on which tools can be placed. The removable inner tray 126 sits in a top portion of the inner cavity 122, blocking access to a bottom portion of the inner cavity 122. The removable inner tray 126 can be lifted fully out of the storage apparatus to provide access to the bottom portion of the inner cavity 122. A UV light may be located in the upper portion of the inner cavity 122 or the bottom portion of the inner cavity 122. An additional sanitizer may be provided. For example, a utensil cleaning slot 130 is shown on the left side wall 115 in FIG. 1B. This utensil cleaning slot 130 may be a drawer configured to slide or pivot to open and close. The drawer may be sized, shaped, and configured to hold a liquid sanitizing solution such as Barbicide® and tools for dipping in the liquid sanitizing solution.

The embodiment of FIG. 1A, FIG. 1B, and FIG. 2 further includes an extendable and retractable hair tray 132 and a pivotable plug access panel 140 on the front wall 117 configured to hold and manage cords and cover a plurality of electrical ports. In this embodiment, a bracket system 150 is used to secure the storage apparatus 100 to a chair. The bracket system includes two adjustable top brackets 152 configured to fit over the top of a chair and two side brackets 154 configured to fit around the side of a chair. FIG. 1A also depicts one possible position of the retractable handle 160 and wheels 170 provided for ease of transport. As shown in FIG. 1B and FIG. 2, in this embodiment, the battery is integrated into the storage apparatus and features a charging port 182 for connecting the battery, via an electrical cable, to an external outlet for charging. Storage slots 136 are also included in the front in this depiction. In this embodiment, all or a portion of the outer body walls are formed of one or more of iron, nickel, or cobalt and are magnetic.

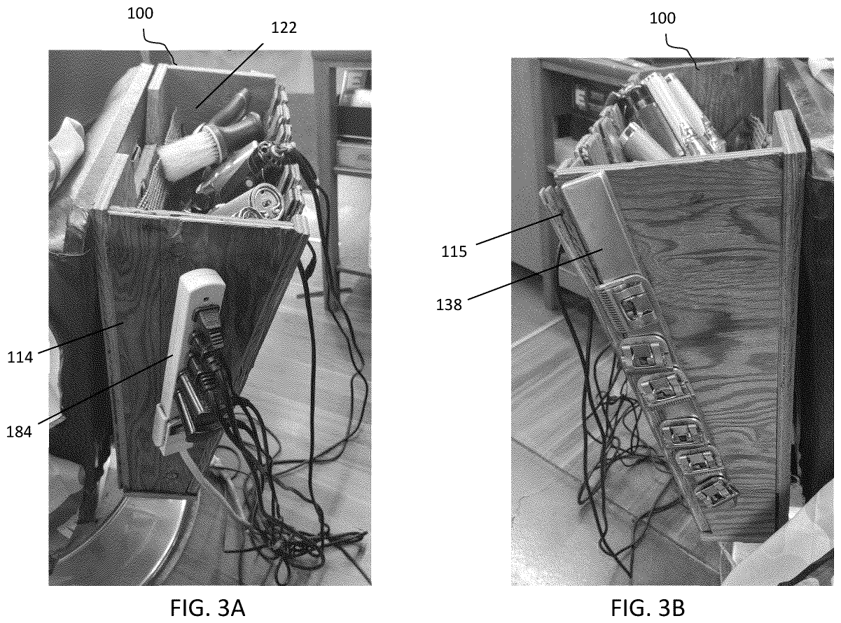

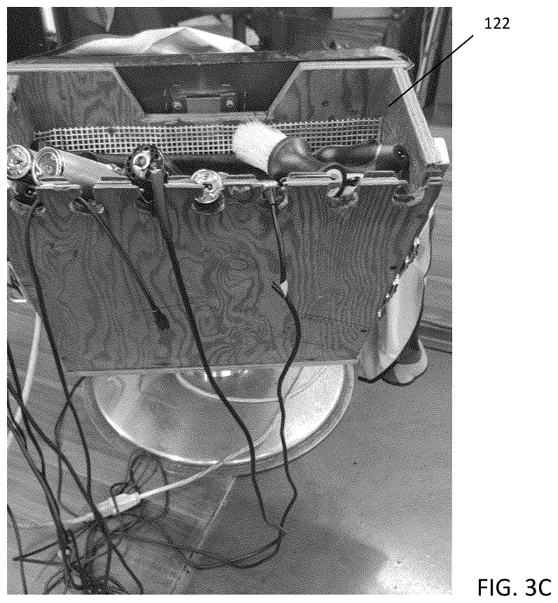

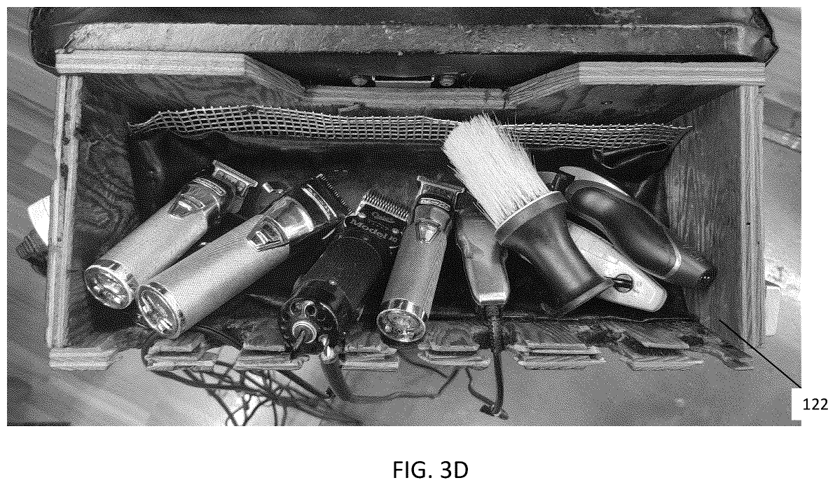

FIGS. 3A-3D feature an early prototype of the storage apparatus 100. In this embodiment, a power strip 184 is secured to the right side wall 114 of the storage apparatus 100 to charge equipment. The power strip 184 may be electrically connectable to a detachable, rechargeable battery that is coupled directly or indirectly to the body 110 of the storage apparatus 100. A magnetic strip 138 is affixed to a left side wall 115 of the storage apparatus 100 and may be used to store magnetic tools and components during grooming services. One embodiment of the central cavity of the storage apparatus is visible in FIG. 3D. The UV light or other sanitizer may be provided at any suitable location within the central cavity, including on an inner surface of the top lid or on an inner surface of any of the walls defining the central cavity.

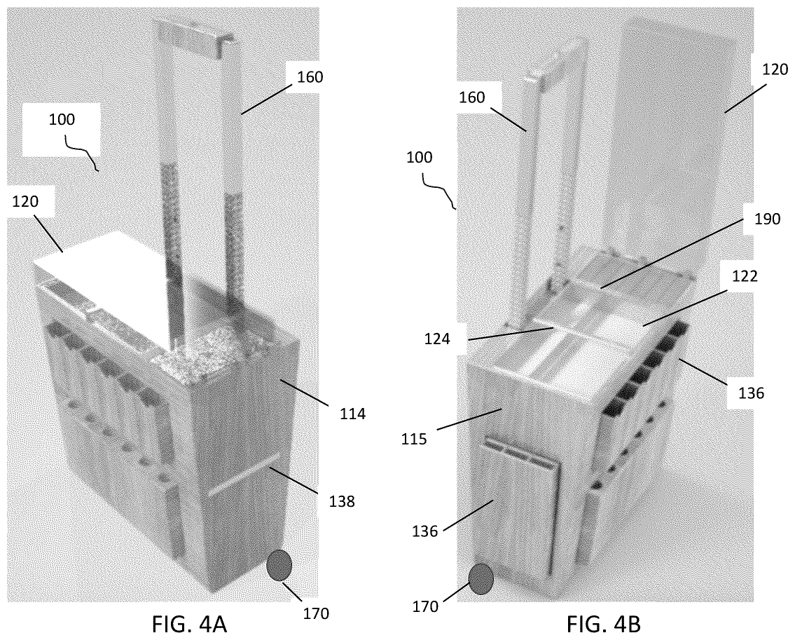

FIGS. 4A-4B feature another embodiment of the storage apparatus 100. This depiction includes the retractable handle 160, which extends upward from and retracts downward into a sheath, sleeve, or casing disposed on or in a back wall of the storage apparatus body. A set of roller wheels 170 is also provided on a bottom portion of the storage apparatus 100. A horizontal magnetic strip 138 is provided on the right side wall 114 of the storage apparatus 100, extra storage compartments 136 are provided on the front side, and a hair tray 132 is provided on the left side 115. The top includes a lid 120 (shown in a closed configuration in FIG. 4A and in an open configuration in FIG. 4B). In the open configuration, the central cavity 122 having a plurality of storage spaces, a divider 124, and a UV light 190 are visible.

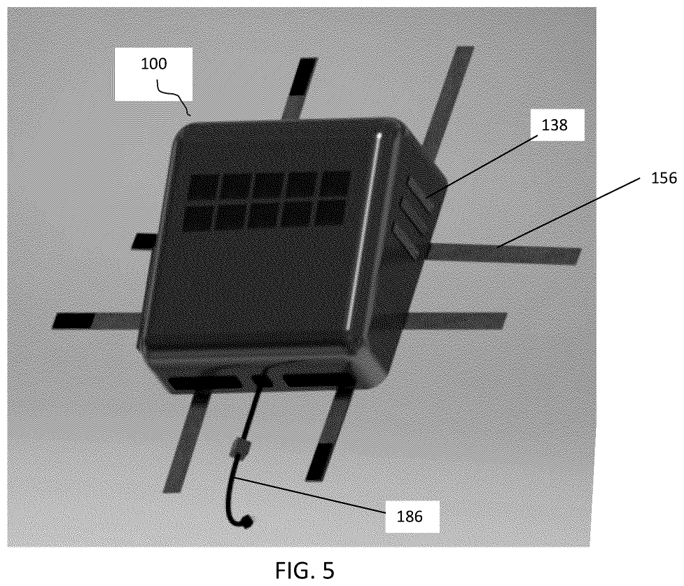

FIG. 5 features another embodiment of the storage apparatus 100. In this embodiment, tension straps 156 are used to secure the storage apparatus 100 to the barber chair or other seat. The battery is a permanent fixture, integrated into the body of the storage apparatus 100. A retractable cord 186 is extendable from a bottom portion of the body for the purpose of charging the battery. This embodiment also features three magnetic strips 138 on the side of the storage apparatus 100 for storage of magnetic tools that do not require a charge.

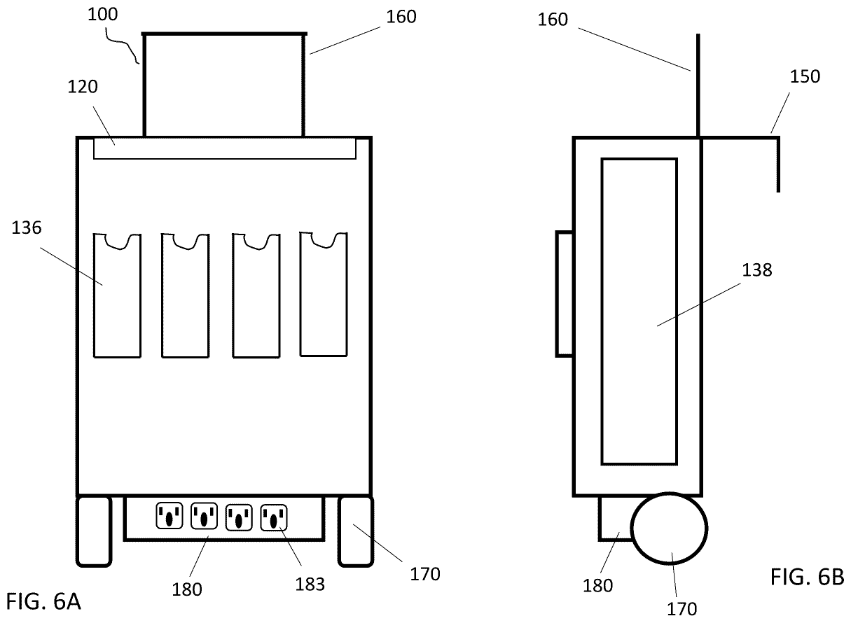

FIGS. 6A-6B feature another embodiment of the portable storage apparatus 100. To facilitate transport, this embodiment includes a set of wheels 170 and a handle 160 (which may or may not be retractable). To facilitate convenient access to grooming tools while a person is providing grooming services, the embodiment includes a bracket system 150 for secure attachment of the storage apparatus 100 to a chair. It also includes a lid 120 on a top portion covering an opening to the central cavity, storage pockets 136 on a front wall, and a magnetic strip 138 on a side wall. It further includes a rechargeable battery 180 coupled to an outer surface (e.g., a bottom wall) of the storage apparatus body. Several ports 183 are positioned on a surface of the battery 180 for providing electrical current from the battery 180 to electric grooming tools. In this particular embodiment, four ports 183 are provided, however, in other embodiments, one, two, three, four, five, or more charging ports 183 may be provided.

In another embodiment, the device comprises a UV shielding device, which includes a sanitizing interface having a top surface arranged to support Barber Supplies positioned above the sanitizing interface. The device also including an adjustable UV shield positioned adjacent to the sanitizing interface and arranged to conform substantially to a shape of the Barber Supplies positioned above the sanitizing interface, where the UV shield includes a first perimeter positioned in proximity laterally to a perimeter of a Barber Clipper. The portable electronic sanitizing device has an assembly including a UV device, a base comprising a device battery for powering the UV device, and a wireless charge receiving system. A cover lift system for a Portable Barber Case includes a lifter handle having an upper arm. The upper arm is configured for operative connection to a cover of a Portable Barber Case. An adjuster mechanism associated with the lifter handle may also be provided for selectively adjusting a length of the lifter handle to allow for use of the lifter handle with the Portable Barber Case.

In various embodiments, the storage apparatus includes one or more of the following features: a UV Sanitizer, Portable UV Sterilizer, Sterilizer Using UV Light, Sterilizes Virtually Anything in Minutes with No Cleaning Required, Touch Screen Control, Wireless Charging System, Multi Device Charging Station—5V 1.3 A Charger with Micro USB Cable, PowerStation Wireless—External Battery Charger, Charging Hub 5 Port, and an Ultraviolet Sterilization Box.

Unless otherwise defined, each technical or scientific term has the same meaning as commonly understood by one of ordinary skill in the relevant art.

As used in the specification and claims, the singular form “a”, “an” and “the” include both singular and plural references unless the context clearly dictates otherwise. For example, the term “a magnetic strip” is contemplated to include a plurality of magnetic strips. At times, the claims and disclosure may include terms such as “a plurality,” “one or more,” or “at least one;” however, the absence of such terms is not intended to mean, and should not be interpreted to mean, that a plurality is not conceived.

As used in the specification and claims, the term “comprising” or “comprises” is intended to mean that the devices, systems, and methods include the recited elements and may additionally include any other elements. Terms such as “including” and “having” are intended to be open terms synonymous with “comprising” and should be interpreted to respectively mean, for example, “including but not limited to” and “having at least.”

The foregoing has included detailed descriptions of some features of the technology. Features of the various embodiments can be arranged, combined, and designed in a variety of different configurations, all of which are explicitly contemplated and form part of this disclosure. For example, any of the components described or depicted in one embodiment may be combined or interchanged with components, or excluded, from other embodiments. In light of the provided illustrations and examples, it will be readily apparent to those of ordinary skill in the art that numerous changes and modifications may be made without departing from the spirit or scope of the appended claims.

1. A storage apparatus for grooming equipment comprising:
a body comprising a plurality of walls,
wherein the body defines a central cavity and has an opening to access the central cavity, and
wherein at least a portion of a first wall of the body is magnetic;
a handle coupled to the body;
a plurality of wheels coupled to the body;
a movable lid coupled to the body and configured to cover the opening to the central cavity;
a divider within the central cavity;
a rechargeable battery attached to or integrated into the body; and
a securement feature for securing the storage apparatus to a chair.

2. The storage apparatus of claim 1, wherein the handle is accessible from a top side of the body.

3. The storage apparatus of claim 1, wherein the handle is retractable into a sheath on or in a rear wall of the body.

4. The storage apparatus of claim 1, wherein the opening to the central cavity and the lid are on a top side of the body.

5. The storage apparatus of claim 1, wherein the wheels are positioned on a bottom side of the body.

6. The storage apparatus of claim 1, wherein the divider is removable and positionable at different locations within the central cavity.

7. The storage apparatus of claim 1, wherein the storage apparatus comprises a plurality of dividers, and wherein the plurality of dividers and the plurality of walls together define a plurality of storage compartments for grooming tools within the central cavity.

8. The storage apparatus of claim 1, wherein the battery is detachable.

9. The storage apparatus of claim 1, wherein the battery is positioned on a bottom side of the body.

10. The storage apparatus of claim 1, further comprising a sanitizer within the central cavity.

11. The storage apparatus of claim 10, wherein the sanitizer comprises a UV light.

12. The storage apparatus of claim 1, further comprising an electrical charging means disposed within said central cavity.

13. The storage apparatus of claim 12, wherein the electrical charging means comprises one or more of: an electrical port, and a wireless charging coil.

14. The storage apparatus of claim 1, further comprising an electrical port on a surface of the body or a surface of the rechargeable battery.

15. The storage apparatus of claim 1, further comprising a storage pocket on an outer surface of the body.

16. The storage apparatus of claim 1, wherein the securement feature comprises at least one of: a bracket, a hook, and a strap.

17. The storage apparatus of claim 1, wherein the first wall of the body comprises at least one of: a left side wall, a right side wall, and a front wall.

18. The storage apparatus of claim 1, wherein the magnetic portion of the first wall comprises a magnetic strip.

19. The storage apparatus of claim 1, wherein an entirety of the first wall is magnetic.
